(19) United States
(12) Patent Application Publication
(10) Pub. No.: US 2010/0168784 A1
(43) Pub. Date: Jul. 1, 2010

(54) Laparoscopic Vaginal Cuff Occluder

(76) Inventor: Terri Lynne Behrman Pustilnik, Houston, TX (US)

Correspondence Address: The McBride Law Firm, P.C., 4265 San Felipe, Suite 1100, Houston, TX, 77027, US

(21) Appl. No.: 12/639,051

(22) Filed: Dec. 16, 2009

Related U.S. Application Data
(60) Provisional application No. 61/201,908, filed on Dec. 16, 2008.

(51) Int. Cl. A61M 29/02

(52) U.S. Cl. 606/193

(57) ABSTRACT

Embodiments described herein comprise an apparatus and method for preventing fluid from flowing through the vagina. The apparatus comprises an introducer for inserting into the patient's vagina. The apparatus may further comprise an expandable device coupled to the introducer. The expandable device may be configured to expand into a substantially sealing engagement with an inner wall of the patient's vagina once the introducer is located within the patient's vagina. The expandable device may be capable of maintaining a fluid pressure in the abdominal cavity of the patient after a fluid connection has been made with the vagina and the abdominal cavity.

### CROSS-REFERENCE TO RELATED APPLICATION

This application claims the priority benefit of U.S. provisional patent application No. 61/201,908, titled “Laparoscopic Vaginal Cuff Occluder,” filed Dec. 16, 2008 with the inventor Terri Pustilnik. This related application is hereby incorporated by reference in its entirety.

### BACKGROUND

Embodiments of the inventive subject matter generally relate to the field of surgical tools, and more particularly to an apparatus and method for preventing fluid flow through a vagina during a laparoscopic procedure.

Laparoscopic surgical equipment has allowed surgeons to remove the uterus and/or ovaries through less invasive procedures. The laparoscopic surgery eliminates the need for a long abdominal incision, thereby reducing recovery time of the patient. During a laparoscopic hysterectomy a surgeon passes a laparoscope trans-abdominally through an abdominal incision. A pneumoperitoneum condition may be created in the abdominal cavity by insufflating CO2 gas into the abdominal cavity during the surgery. The pneumoperitoneum condition allows the surgeon to better visualize and access the internal organs, for example the uterus, of the patient by enlarging the abdominal cavity. The uterus, the ovaries, the fallopian tubes, the lymph nodes and/or other structures may then be cut from the body for removal from the body. A portion of the vagina, for example the vaginal cuff, may be cut open during the surgery. Upon cutting the vagina, the pneumoperitoneum condition is lost due to escape of CO2 gas through the vagina. The loss of the CO2 gas causes loss of intra-abdominal pressure. The uterus, the ovaries and/or the lymph nodes may then be removed from the body through the vagina. The cut portion of the vagina may then be sutured back together, or sealed. The surgeon may then remove the laparoscopic tools from the abdomen when the operation is completed.

### SUMMARY

Embodiments of the invention include a surgical tool having an introducer, wherein the introducer is configured to insert into a vagina. The surgical tool further comprises an expandable device couplable to a portion of the introducer, wherein at least a portion of the expandable device is configured to expand into a wall of the vagina. The surgical tool further comprises an actuator for expanding the expandable device and a handle for manipulating the surgical tool wherein the expandable device is configured to substantially seal the vagina during a surgical procedure thereby preventing fluids from flowing past the expandable device and the wall of the vagina.

Embodiments of the invention include a method for substantially preventing fluid from flowing through a vagina during surgery. The method comprises filling a portion of the abdominal cavity with a fluid and inserting an introducer of a surgical tool into the vagina. The method further comprises expanding an expandable device coupled to the introducer into a wall of the vagina. The method further comprises cutting a portion of the vagina thereby fluidly coupling the abdominal cavity with vagina and preventing the fluid from flowing out of the vagina.

Embodiments of the invention include a method for substantially preventing fluid from flowing through a vagina during surgery. The method includes filling a portion of the abdominal cavity with a fluid. The method further includes selecting a surgical tool having an introducer with an outer perimeter and inserting an introducer of a surgical tool into the patient's vagina. The method further includes sealing the vagina with at least a portion of the introducer and cutting a portion of the vagina thereby fluidly coupling the abdominal cavity with vagina. The method further includes preventing the fluid from flowing out of the vagina.

Embodiments of the invention include a surgical tool having an introducer. The introducer is configured to insert into a patient's vagina. The surgical tool further comprises a balloon coupled to the introducer and configured to expand into engagement with an inner wall of the vagina. The surgical tool further comprises an actuator configured to inflate the balloon from an unexpanded condition to the expanded condition and a handle for manipulating the surgical tool.

### BRIEF DESCRIPTION OF THE DRAWINGS

The present embodiments may be better understood, and numerous objects, features, and advantages made apparent to those skilled in the art by referencing the accompanying drawings.

FIG. 1 depicts a diagram illustrating a schematic view of a surgical tool in an embodiment.

FIG. 2 depicts a diagram illustrating a schematic view of a body of the surgical tool in an embodiment.

FIG. 3A depicts a diagram illustrating a schematic view of a surgical tool in an embodiment.

FIG. 3B depicts a diagram illustrating a schematic view of a surgical tool in an embodiment.

FIG. 4 depicts a diagram illustrating a surgical tool kit in an embodiment.

### DESCRIPTION OF EMBODIMENT(S)

The description that follows includes exemplary apparatus, methods, techniques, and instruction sequences that embody techniques of the present inventive subject matter. However, it is understood that the described embodiments may be practiced without these specific details.

Embodiments described herein comprise an apparatus and method for maintenance of a pneumoperitoneum in the abdominal cavity during a laparoscopic procedure. During a laparoscopic procedure a pneumoperitoneum, or positive fluid pressure, is maintained in the abdominal cavity in order to allow the surgeon to easily visualize and access the pelvic organs such as the ovaries, the uterus, the fallopian tubes, bladder, pelvic floor, lymph nodes, and the like. With the pneumoperitoneum maintained, the surgeon may introduce one or more laparoscopic tools into the abdomen to perform a surgical procedure without the need for cutting a large incision in the patient. During the procedure it may be necessary to create a disruption between the vaginal vault and the abdominal cavity. The disruption may be created by cutting the vaginal cuff thereby creating an opening and a fluid path between the vaginal vault and the abdominal cavity. Although the disruption is described as being made at the vaginal cuff, it should be appreciated that any suitable location may be used for the incision including, but not limited to the vaginal apex, the cervico-vaginal junction, and the like. The surgeon may use the opening in the vagina to remove organs from the body through the vagina. By removing the organs through the vagina, the need for a large abdominal incision is alleviated, thereby minimizing the recovery time and post operative pain of the patient. However, when a disruption is made between the vaginal vault and the abdominal cavity, the pneumoperitoneum will be lost if the vagina is not sealed, or substantially sealed, during the procedure.

In order to seal the vagina, a surgical tool may be inserted into the vagina. The surgical tool may have an introducer configured to insert into the vagina and a handle for manipulating the surgical tool. The surgical tool may further have an expandable device coupled to the introducer and an actuator for actuating the expandable device. The expandable device may be configured to expand into a substantially sealing engagement with an inner wall of the vagina. Thus, during the surgical procedure, the introducer may be inserted into the vagina and a user may actuate the actuator. The actuator may expand the expandable device into a substantially sealing engagement with the inner wall of the vagina. With the surgical tool sealing the vagina, the surgeon may create a disruption between the abdominal cavity and the vaginal vault without compromising the pneumoperitoneum. The surgeon may then complete the surgical procedure, for example removing the uterus and/or ovaries into the vaginal vault and then repairing the disruption in the vagina while maintaining the surgical tool in a sealing engagement with the vaginal wall. With the disruption repaired, the surgeon may use the actuator to disengage the expandable device from a sealing engagement with the inner wall of the vagina. The surgical tool may then be removed from the vagina, and the organs may be removed from the body. The tool may be reinserted into the vagina after the organs are removed, allowing the pneumoperitoneum to be regained and the procedure to be continued laparoscopically. After the vaginal defect is repaired the surgical tool is removed.

In an additional embodiment, the surgeon may have a kit having a number of introducers varying in size. The surgeon may choose the introducer that will best fit the vagina of the patient. In an older woman the vagina may be substantially narrowed or atrophic and consequently require a smaller device. Conversely, a younger woman may require a wider introducer in order to create a better seal. The user, or surgeon, may then insert the introducer into the vagina in a similar manner as described above, without the need for the expandable device.

Another function of the surgical tool allows the surgeon to better delineate the vaginal cuff if the uterus and cervix have been previously removed. By applying upward force to a handle of the surgical tool, the introducer advances up against the closed vaginal apex. The pressure of the introducer against the vaginal apex may make the vaginal wall more apparent for a given procedure. In this manner, the surrounding organs that may over lay the vagina such as ovaries/fallopian tubes/colon/bladder and diseases such as pelvic adhesions and endometriosis can be more safely addressed. Surgeries where it may be necessary to identify the vaginal wall may include; the removal of the ovaries and the fallopian tubes, release of adhesions, release of endometriosis, and assisting in a bladder suspension or suspension of the vaginal vault in the case of vaginal prolapse or uterine prolapse. In addition, suture and mesh may need to be attached to the vagina during a procedure whereby the vagina is attached to the back of the pelvis at the sacrum in a procedure called a sacrocolpopexy. The surgical tool will more safely allow for the correct placement of the suture and mesh. The instrument could be also be inserted into the rectum during laparoscopic prostate surgery to show the rectum separate from the prostate.

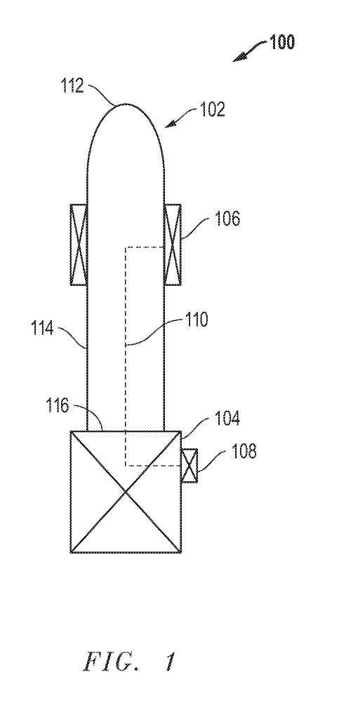

FIG. 1 depicts a surgical tool 100 configured to substantially maintain a pneumoperitoneum in an abdominal cavity when a disruption between the abdominal cavity and the vaginal vault is made. The surgical tool 100 may include an introducer 102, a handle 104, an expandable device 106, and an actuator 108 for actuating the expandable device 106. The actuator 108 may include a communication device 110 for communicating with the expandable device 106. During a surgical procedure, a surgeon may create a pneumoperitoneum in an abdominal cavity and remove one or more organs of a patient. If the uterus and cervix are in place, two devices may be used to manipulate the cervix and the uterus. These devices are a rumi, or a V-care (not shown). Once the organ or organs, for example, the uterus is detached from its blood supply and their attachments, the organ is removed through the vagina. The surgical tool 100 may then be inserted into the vagina to maintain the pneumoperitoneum after removal of the uterus/cervix. This may allow the user to perform further surgical procedures, for example removing the lymph nodes, performing biopsies, and suturing the vaginal cuff closed. If the uterus has already been removed prior to the surgery then the surgical tool 100 may be used to maintain the pneumoperitoneum from the beginning of the procedure if the vagina is opened. It can also be used to delineate the vagina and better visualize the anatomy if the vagina is not disrupted. Further, if at the beginning of the procedure the cervix is too scarred to allow the rumi, or V-care to be used, the surgical tool may be used initially even when the uterus is present.

The surgeon may then want to create a disruption in the vaginal vault in order to remove the one or more organs through the vagina. Prior to creating the disruption the surgeon may grab the handle 104 of the surgical tool 100 and insert the introducer 102 into the patient's vagina. With the introducer 102 inserted into the patient's vagina the surgeon may then use the actuator 108 in order to activate the expandable device 106. The activation of the expandable device 106 causes the expandable device 106 to increase in diameter and thereby into a substantially sealing engagement with an inner wall of the vagina. The engagement of the inner wall with the expandable device 106 allows the surgeon to create a disruption between the vaginal vault and the abdominal cavity without the loss of the pneumoperitoneum. The substantial seal between the expandable device 106 and the inner wall of the vagina may substantially maintain the pneumoperitoneum after the disruption. The surgeon may then move one or more of the patient's organs into the vaginal vault and repair the disruption. Further still, the surgeon may remove one or more organs, for example the uterus and cervix, then reinsert the introducer 102 and expand the expandable device 106 in order to perform more procedures. With the disruption repaired, the surgeon may deactivate the expandable device 106 thereby releasing the seal. The surgeon may then grip the handle 104 and remove the surgical tool 100 from the patient's vagina. The surgeon may then complete the procedure. It should be appreciated that the surgeon may be any person capable of performing any portion of the medical procedure including, but not limited to, a doctor, a resident, a registered nurse, a nurse in training, a medical technician, a surgical technician, a physician's assistant, a medical student, and the like.

The introducer 102 may include a nose 112, and a body 114. The nose 112 may be configured to assist in the insertion of the introducer 102 into the vagina. As shown, the nose 112 has a rounded shaped which has a continuously curved surface going from the terminal end of the introducer 102 to the body 114. The rounded shape may continuously increase the diameter of the nose 112 until the diameter of the body 114 is matched. The nose 112 with the rounded shape enters the vagina with a small surface area, or diameter, and gradually increases in diameter as the introducer 102 is inserted into the vagina. Thus, the nose 112 of the introducer 102 may allow the surgeon to insert the introducer 102 into the vagina with minimal trauma to the vagina. Although the nose 112 is described as having a rounded shape, it should be appreciated that the nose may have any suitable shape for inserting the introducer 102 into the vagina. Once inserted, the introducer 102 may be visualized via the abdominal cavity for proper positioning with the aide of the laparoscopic camera as the vaginal vault has been opened following detachment of the uterus and cervix from the vagina.

The nose 112 may further be used to locate the vaginal cuff, or location for creating the disruption, during the procedure. In this example, the surgeon may insert the introducer 102 into the vagina until the nose 112 is engaged with the vaginal cuff if the vagina has not been disrupted. By applying upward and/or inward pressure from the handle, the introducer 102 may stretch the vaginal wall making the vagina readily visible. The nose 112 may be detectable from the abdominal cavity thereby assisting the surgeon in locating the correct location for the disruption in the vagina to be made. If there are other organs overlaying the vaginal cuff, such as the bladder, bowel, ovaries or tubes, they can be carefully dissected off before exposing the vaginal apex and entering the vaginal vault.

The body 114 of the introducer, as shown, has a cylindrical shape with a substantially similar diameter the entire length of the body. The body 114 may be configured to couple the expandable device 106 to the surgical tool 100. As shown in FIG. 1, the expandable device 106 is attached to the exterior surface of the body 114. The body 114 may be a solid member, or may include a hollow interior. Further, the body 114 may include a port, or channel, configured to allow the communication device 110 to travel through the body 114. The body 114 may be any suitable length capable of delivering the expandable device 106 into the vagina. The body 114 may have substantially the same diameter for the entire length, or have a varying diameter. For example, the body 114 may have a diameter which is wider at the nose 112 and the expandable device 106 and narrows toward the handle, or visa versa. Further, a portion of the body 114 may have a different diameter than another portion of the body 114.

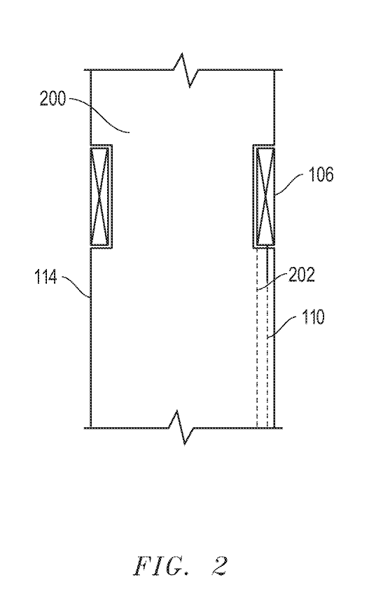

FIG. 2 shows an alternative, or additional, embodiment of the body 114 of the introducer 102. In this embodiment, the body 114 includes a pocket 200 and a channel 202. The pocket 200 may be configured to house all, or a portion, of the expandable device 106 when the expandable device 106 is in the unexpanded position. With the pocket 200 housing the expandable device 106, the expandable device may be protected from inadvertently engaging the vagina before activation. The channel 202 may be configured to protect and/or enclose the communication device 110. As shown in FIG. 2, the channel 202 is a groove which runs in a substantially longitudinal direction along the body 114 in which the communication device 110 may be located. It should be appreciated that the channel 202 may take any suitable shape and form so long as the actuator 108 is capable of activating the expandable device 106.

The handle 104 may be any device capable of coupling to the introducer 102 and being gripped by the surgeon. For example, the handle 104 may simply be an extension of the body 114 which the surgeon may grab when inserting the introducer 102. Further, the handle 104 may be shaped for easier manipulation by the surgeon. The handle 104 may extend beyond the introducer 102 outside the body, when the introducer 102 is used during a procedure. The handle 104 allows for insertion of the introducer 102 into the vagina and for easy manipulation and visualization by the surgeon. In an alternative, or additional embodiment, the handle 104 may bend at an angle relative to the longitudinal axis of the introducer 102. For example, the handle 104 may have a bend that allows a portion of the handle 104 to be at a substantially right angle to the introducer 102. This may allow a portion of the handle 104 to be located above the vagina when the introducer 102 is inside the patient's vagina. This may allow for easier access to the handle 104 by the surgeon. Although the handle 104 is described as being at a 90 degree angle to the introducer 102, it should be appreciated that any suitable angle may be used.

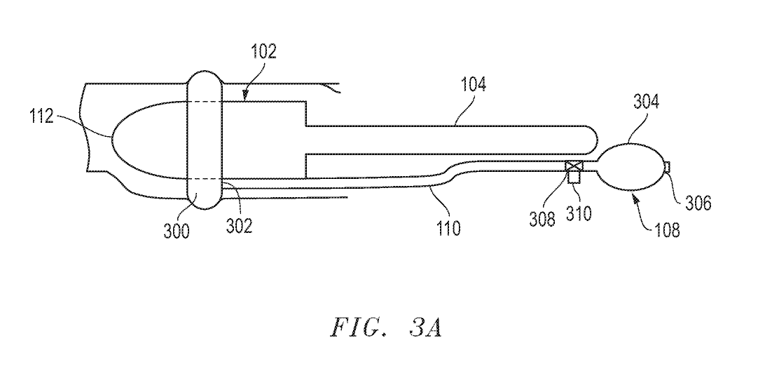

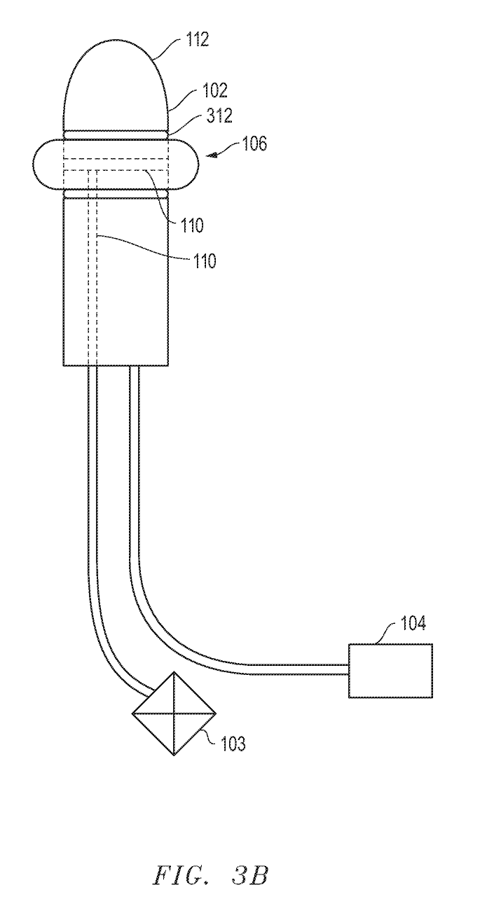

The expandable device 106 may be a balloon 300, as shown in FIG. 3A, in one embodiment. The balloon 300 may secure to the exterior surface of the introducer 102, or the pocket 200, as shown in FIG. 2. The balloon 300 may be any inflatable device capable of expanding radially away from the introducer 102. The balloon 300 may be constructed of any suitable expandable material including, but not limited to, rubber, latex, polychloroprene, a nylon and the like. The balloon 300 may be constructed in a circular, or doughnut shaped, manner with an opening 302, or port. The opening 302 may allow the balloon 300 to couple to the communication line 110 in order to fill the balloon 300. In an uninflated position, the balloon 300 may be sized so that the balloon 300 will securely engage the introducer 102, thereby allowing a minimal portion, or none, of the balloon 300 to extend beyond the outer diameter of the introducer 102. In the uninflated position, the introducer 102 with the balloon 300 may easily be inserted into the vagina. In the inflated position, the balloon is filled with a fluid thereby expanding the balloon 300 radially into engagement with the inner wall of the vagina as shown in FIG. 3A. The fluid may be introduced into the balloon 300 through the opening 302 via the communication line 110, as will be discussed in more detail below. Although the communication line 110 is shown as a separate component than the balloon 300, it should be appreciated that the communication line 110 may be integral with the balloon 300. The fluid used to fill the balloon 300 may be any suitable fluid including, but not limited to, air, helium, hydrogen, oxygen, gas, water, saline, liquid, and the like.

In the inflated position, the balloon 300 may substantially prevent fluid from flowing past the introducer 102 in the vagina. Thus, in the inflated position, the balloon 300 acts like a plug in the vagina. The inflated balloon 300 may further act to prevent the inadvertent removal of the introducer 102 from the vagina. For example, the inflated balloon 300 may slightly compress the portion of the vagina in which balloon 300 engages. This compression may cause the vagina to have a slightly larger diameter at the location of the balloon 300. Thus, the narrower portion of the vagina on either side of the balloon 300 may prevent the introducer from inadvertently moving in either longitudinal direction during the surgical procedure. Further, the material of the balloon 300 may further prevent the inadvertent movement of the introducer 102 during the surgical procedure. For example, a rubber or latex material may tend to stick to the inner wall of the vagina, thereby preventing longitudinal movement of the introducer 102 during the surgical procedure.

Although the expandable device 106 is described as a balloon 300, it should be appreciated that the expandable device may be any suitable device capable of being expanded into the inner wall of the vagina to result in a seal. For example, the expandable device 106 may be an elastomeric material that surrounds the introducer 102. The elastomeric material may either be mechanically moved radially away from the introducer 102 and into engagement with the inner wall of the vagina, or may be longitudinally compressed thereby forcing the elastomeric material to move radially away from the introducer 102 and into engagement with the inner wall of the vagina. Further, the expandable device 106 may be any suitable expandable device including, but not limited to, a swelling elastomeric material, a foam, a sponge and the like.

FIG. 3B shows an additional, or alternative, embodiment of the surgical tool 100. In this embodiment, the balloon 300 may have an open end and a closed end and may fit over the introducer 102 like a sock would fit on a foot, or a condom would fit on a penis. The balloon 300 may be secured over the entire introducer 102 or a portion thereof. Once the balloon 300 is secured over the introducer 102, it may be necessary to seal a portion of the balloon 300 to the introducer 102. To this end, one or more o-rings 312, or seals, may be placed over the balloon 300 and introducer 102. The introducer 102 may have one or more o-ring notches which circumscribe all, or a portion of the introducer 102. The o-ring notches may be configured to secure the o-rings 312 in a particular location on the introducer 102. For example, the o-ring notches on the introducer 102 as shown in FIG. 3B are located radially inside where the o-rings 312 are located. The o-ring notches may be any suitable device for securing the o-rings 312 in a longitudinal location along the introducer 102 including, but not limited to, a circumferential indentation, one or more ridges, one or more bosses, one or more lips, and the like.

The o-rings 312 may secure the balloon 300 to the introducer 102 thereby sealing a portion of the balloon 300 when the balloon 300 is inflated. The o-ring 312 may be made from an elastic material. For example, the o-ring 312 may be an elastic member that has a slightly smaller inner diameter than the outer diameter of the introducer 102, when the o-ring 312 is in an unexpanded position. Once the balloon 300 is over the introducer 102, the surgeon may expand the o-ring 312 and secure it to the location of the o-ring notches. Because the outer diameter of the introducer 102 is larger than the inner diameter of the unexpanded o-ring 312, the elasticity of the o-ring 312 may secure the balloon 300 to the introducer 102.

In FIG. 3A, the communication line 110 may run through the introducer 102 and into the portion of the balloon 300 that is sealed. Once the introducer 102 is secured in the vaginal, the surgeon may actuate the actuator 108 thereby inflating the sealed portion of the balloon 300 into a substantially sealed engagement with the inner wall of the vagina. Although, the balloon 300 is shown as being inflated between two of the o-rings 312, it should be appreciated that the balloon 300 may only require one o-ring 312, and thereby inflate the portion between the o-ring 312 and the sealed end of the balloon 300. Further, there may be more than two o-rings 312 and more than one inflated portion of the balloon 300.

The actuator 108, as shown in FIG. 3A, is a bulb 304. The bulb 304 may be a typical inflation bulb. In one embodiment, the bulb 304 may include an air intake one way valve 306, an air exit one way valve 308, and a release valve 310. In an undisturbed state, the bulb 304 is in an expanded position and full of a fluid. When the surgeon wishes to inflate the balloon 300, the surgeon squeezes the bulb 304. As the surgeon squeezes the bulb 304, the air intake one way valve 306 prevents air from exiting the bulb 304 while the air exit one way valve 308 allows the air to flow toward the balloon 300. The air flowing toward the balloon 300 inflates the balloon 300. The surgeon may then release the bulb 304. The bulb 304 may be configured to automatically return to its bulbous shape upon release. While returning to its bulbous shape, the exit one way valve 308 prevents air in the balloon 300 from flowing back into the bulb 304. The air intake one way valve 306 allows fluid from outside the bulb 304 to be pulled into the bulb 304. Once the bulb 304 has substantially returned to its bulbous shape, the surgeon may squeeze the bulb 304 again. This step may be repeated until the balloon 300 has the desired fluid pressure. When the surgical procedure is complete, the surgeon may open the release valve 310 in order deflate the balloon 300.

Although described as a bulb 304, it should be appreciated that any suitable actuator for expanding the expandable device 106 may be used including, but not limited to, a Luer Lock tip syringe, a pump, a compressed fluid source, a servo, and the like.

The communication line 110 as shown in FIG. 3A is a flexible tubing. The flexible tubing may be similar to, or integral with, the balloon 300 material. Further, the communication line 110 may be any suitable device capable of expanding the expandable device 106 including, but not limited to, plastic tubing, a hard wire line, one or more bore holes in the introducer 102, or any combination thereof, and the like.

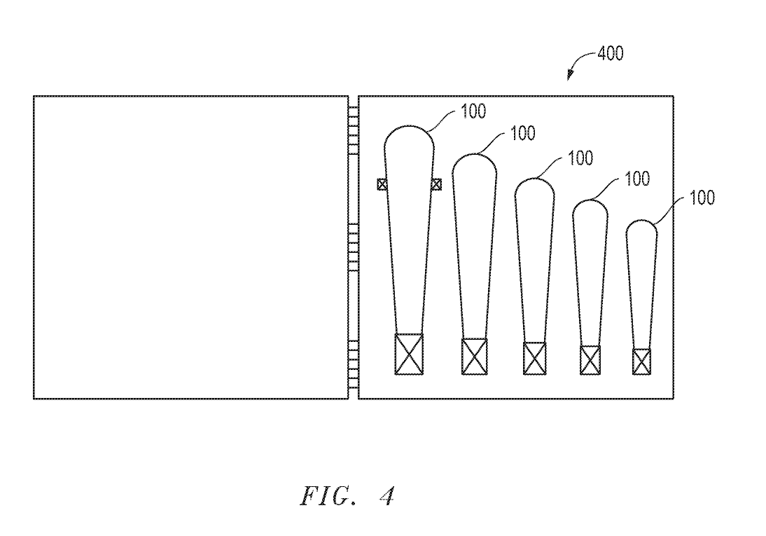

In an alternative or additional embodiment, there may be several surgical tools 100 in a kit 400, as shown in FIG. 4. The surgical tools 100 may be sized in order to fit easily into a variety of sized vaginas. Thus, the surgeon may choose the surgical tool 100 that is sized to match the size of the patient's vagina. The kit 400 may allow the surgical tool 100 to work without the need for the expandable device 106. In this example, the surgical tool 100 may be sized to have a slightly larger outer diameter than the inner diameter, or width of the inner wall of the vagina. Thus, the surgeon may choose the surgical tool 100 with a slightly larger outer diameter than that required by the patient. The surgeon may then insert the introducer 102 of the chosen surgical tool 100 into the vagina of the patient. Due to the larger size of the surgical tool 100 than the patient's vagina, the tool may maintain the fluid pressure in the vagina even after vagina has been disrupted. The introducer 102 may be tapered in a similar manner as described above in order to prevent the introducer 102 from inadvertently becoming dislodged from the vagina. When the procedure is complete, the surgeon may remove the introducer 102 from the vagina. The surgeon may then sterilize the surgical tool 100 and return the surgical tool 100 to the kit 400.

The surgical tool 100 may be constructed of any suitable material or combination of materials. In one embodiment, the introducer and handle may be constructed of any suitable material that may be easily sterilized including, but not limited to, a surgical steel, a plastic, aluminum, acrylic and/or any combination thereof. The expandable device 106 may be a disposable element that may be removed from the surgical tool 100 after the procedure is performed. For example, the expandable device 106 may be a rubber, a plastic or latex material, which is inexpensive. Further, the entire surgical tool 100 may be constructed of an inexpensive material that may be disposed of after the procedure, for example a plastic material. Further, any combination of materials described herein may be used to construct the surgical tool 100.

In operation, the surgeon may create one or more cuts, or puncture, in the patient's abdomen. The surgeon may insert one or more laparoscopic tools into the patient's abdomen through the puncture(s). The surgeon may further create a pneumoperitoneum condition in the abdomen, by filling the abdomen with a positive fluid pressure. The pneumoperitoneum allows the surgeon to operate in the abdominal cavity with more freedom. The surgeon may then severe one or more organs, portions of organs, or tumors, from the patient's body. The organs may be any organ may be any suitable for removal including, but not limited to, an ovary, a uterus, the cervix, a fallopian tube, a portion of the liver, a kidney, the omentum, the peritoneum, the appendix, the gallbladder, the bowel, portion of the stomach, the spleen, and the like. With the organ severed from the body, the surgeon may need to remove the organ from the body; however, the punctures may be too small to allow the organs through. Thus, the surgeon may use the surgical tool 100 in order remove the organs through an alternate opening, or the vagina In some embodiments the uterus and/or cervix may be removed through the vagina prior to using the surgical tool 100. Once the cervix and/or uterus are removed the user would insert the surgical tool as described herein. The surgeon may secure the balloon 300 to the outer surface of the introducer 102. The surgeon may then insert the introducer 102 into the vagina. The surgeon may insert the introducer 102 until the nose 112 engages the apex of the vagina, or cervix of the patient assuming the uterus is gone but the cervix was left in place as in a supracervical hystectomy procedure. With the nose engaged with the cervix, the surgeon may be able to locate the vaginal cuff more precisely from the abdominal cavity. The surgeon may then actuate the actuator 108. The actuator 108 may send fluid through the communication line 110 in order to expand the expandable device 106, for example the balloon 300. The expandable device 106 may then engage the inner wall of the vagina thereby substantially sealing the vagina. The surgeon may then cut the vaginal cuff from inside the abdominal cavity. The incision may be large enough to pass the organs past the vaginal cuff and into the vagina. With the incision made, the fluid pressure from the abdominal cavity may try to escape the body through the vagina. However, the expandable device 106 prevents the fluid from flowing past the surgical tool 100 and out of the vagina. The surgeon may then move all of the organs to be removed into the vagina. The surgeon may then stitch up and/or seal the vaginal cuff thereby preventing fluid from the abdomen from flowing into the vagina. The surgeon may then close, or suture the abdominal incisions. Once the vaginal cuff is sealed, the surgeon may deactivate the actuator 108 and deflate the expandable device 106. The surgeon may then remove the introducer 102 from the vagina.

In an additional embodiment, the organs are placed in a special plastic bag called an endobag (not shown). A string at the top of the bag is pulled tight to close it and the bag is brought down to the pelvic area for removal. In this embodiment, the surgical tool 100 may be removed, then the organs in the bag are removed through the vagina by the surgeon who is standing down below, between the patients legs and pulls them out through the vagina. When the bag is removed there may be a necessary deflation of pneomoperitoneum during actual removal of the organs through the vaginal opening. The deflation is temporary and the surgical tool 100 may be reinserted to restore the pneomoperitoneum and continue with the procedure.

While the embodiments are described with reference to various implementations and exploitations, it will be understood that these embodiments are illustrative and that the scope of the inventive subject matter is not limited to them. Many variations, modifications, additions and improvements are possible.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

What is claimed is:

1. A surgical tool, comprising:
an introducer, wherein the introducer is configured to insert into a vagina;
an expandable device couplable to a portion of the introducer, wherein at least a portion of the expandable device is configured to expand into a wall of the vagina;
an actuator for expanding the expandable device; and
a handle for manipulating the surgical tool and wherein the expandable device is configured to substantially seal the vagina during a surgical procedure thereby preventing fluids from flowing past the expandable device and the wall of the vagina.

2. The surgical tool of claim 1, wherein the expandable device is a balloon.

3. The surgical tool of claim 2, wherein the balloon is configured to be removed from the surgical tool and disposed of after the surgical procedure.

4. The surgical tool of claim 1, wherein the actuator is a bulb configured to pump pneumatic fluid into the balloon.

5. The surgical tool of claim 1, wherein the actuator is a luer lock configured to inject pneumatic fluid into the balloon.

6. The surgical tool of claim 1, wherein the introducer further comprises a rounded nose configured to assist in the insertion of the introducer into the vagina.

7. A method for substantially preventing fluid from flowing through a vagina during surgery, comprising:
filling a portion of the abdominal cavity with a fluid;
inserting an introducer of a surgical tool into the vagina;
expanding an expandable device coupled to the introducer into a wall of the vagina;
cutting a portion of the vagina thereby fluidly coupling the abdominal cavity with vagina; and
preventing the fluid from flowing out of the vagina.

8. The method of claim 7, further comprising removing the introducer from the vagina and removing the expandable device.

9. The method of claim 8, further comprising sterilizing the surgical tool.

10. The method of claim 9, further comprising coupling a sterile expandable device to the surgical tool.

11. The method of claim 7, further comprising identifying the vaginal cuff with the nose of the introducer.

12. A method for substantially preventing fluid from flowing through a vagina during surgery, comprising:
filling a portion of the abdominal cavity with a fluid;
selecting a surgical tool having an introducer with a outer perimeter which is larger than a patient's vagina;
inserting an introducer of a surgical tool into the patient's vagina;
sealing the vagina with at least a portion of the introducer;
cutting a portion of the vagina thereby fluidly coupling the abdominal cavity with vagina; and
preventing the fluid from flowing out of the vagina.

13. The method of claim 12, wherein selecting a surgical tool further comprises selecting the surgical tool from a kit having a plurality of varying sized introducers.

14. The method of claim 12, further comprising locating a vaginal cuff with a nose of the introducer prior to cutting a portion of the vagina.

15. A surgical tool, comprising:
an introducer configured to insert into a patient's vagina;
a balloon coupled to the introducer and configured to expand into engagement with an inner wall of the vagina;
an actuator configured to inflate the balloon from an unexpanded condition to the expanded condition; and
a handle for manipulating the surgical tool.

16. The surgical tool of claim 15, wherein the actuator further comprises a luer lock.

17. The surgical tool of claim 15, wherein the introducer further comprises a rounded nose.

18. The surgical tool of claim 15, wherein the introducer further comprises a pocket configured to house at least a portion of the balloon when the balloon is in the expanded condition.

19. The surgical tool of claim 18, further comprising a channel for housing a communication line from the actuator to the balloon.

20. The surgical tool of claim 15, wherein the introducer and handle are constructed of a material capable of sterilization and reuse, and the balloon is constructed of a removable and disposable material.
